(19) United States
(12) Patent Application Publication
(10) Pub. No.: US 2019/0119687 A1
(43) Pub. Date: Apr. 25, 2019

(54) XYLOSIDASE HAVING IMPROVED ENZYMATIC ACTIVITY

(71) Applicant: Dongguan APAC Biotechnology CO., Ltd., DongGuan (CN)

(72) Inventors: Ya-Shan Cheng, New Taipei City (TW); Tzu-Hui Wu, New Taipei City (TW); Cheng-Yen Lin, New Taipei City (TW); Hui-Lin Lai, New Taipei City (TW); Cheng-Bin Zheng, New Taipei City (TW); Ting-Yung Huang, New Taipei City (TW); I-Hsuan Lin, New Taipei City (TW); Jian-Wen Huang, New Taipei City (TW); Chun-Chi Chen, New Taipei City (TW); Rey-Ting Guo, New Taipei City (TW)

(21) Appl. No.: 16/157,692

(22) Filed: Oct. 11, 2018

(30) Foreign Application Priority Data
Oct. 25, 2017 (TW) 106136658

(51) Int. Cl. C12N 15/52; C12N 9/24; C12N 15/10

(52) U.S. Cl. CPC C12N 15/52; C12N 9/2402; C12Y 302/01177; C12Y 302/01072; C12N 15/102

(57) ABSTRACT

A xylosidase having improved enzymatic activity is disclosed. The amino acid sequence of the xylosidase is a modified amino acid sequence of SEQ ID NO: 2, wherein the modification is a substitution of phenylalanine at position 35 with glutamate, and/or a substitution of glutamine at position 41 with histidine.

### FIELD OF THE INVENTION

The present invention relates to a xylosidase, and more particularly to a xylosidase having improved enzymatic activity.

### BACKGROUND OF THE INVENTION

Xylan is hemicellulose, which is the major component in plant cell wall, and also the second most abundant polysaccharides on earth. Therefore, the hydrolytic enzymes that degrade xylan are highly attractive and widely applied in many industries for a long time. Xylan is a long chain polysaccharide, which is composed of many pentose xylose units linked by β-1,4-glycosidic bond as a backbone of xylan. In nature, xylan is complex and highly branched heteropolysaccharide which can be decorated by methyl group or acetyl group, even branched by other sugar molecules to form various structures of xylan. Because of this complicated architecture of xylan, the complete degradation of xylan requires different xylanolytic enzymes to work together for decomposing xylan into simple sugars that can be used by organisms.

In general, xylanolytic enzymes can be divided into several groups including endo-β-D-xylanase, β-1,4-xylosidase, arabinase, acetylxylan esterase and α-glucuronidase. Among these enzymes, β-1,4-xylosidase (EC 3.2.1.37) is a crucial enzyme for complete degradation of xylan. It is an exoglucosidase that can hydrolyze the non-reducing ends of xylooligosaccharides into simple sugar xylose.

Since the xylosidase works together with the endo-xylanase and other xylanolytic enzymes, these enzymes can be cooperatively used in many different industries, such as bleaching process in paper industry, dough quality and juice clearance in food industry, animal nutrition in feed industry, even in biofuel production. According to different industrial needs, xylosidase is required to be suitable for different appropriate working conditions. In addition to the protein properties of enzyme, its catalytic efficiency is also the key point for improving industrial enzyme. Higher enzymatic activity represents the cost reduction in the industrial process and further enhances the commercial profit.

Currently, many researches try to obtain better enzymes by either screening in nature or modifying present enzymes. In the present invention, xylosidase is modified by rational design to increase its enzymatic activity, so as to further increase its application potential and economic value in industry.

### SUMMARY OF THE INVENTION

An object of the present invention is to modify a xylosidase by means of structural analysis and site-directed mutagenesis for improving the enzymatic activity of the xylosidase and further increasing its application potential and economic value in industry.

According to an aspect of the present invention, there is provided a xylosidase comprising a modified amino acid sequence of SEQ ID NO: 2, wherein the modification is a substitution of phenylalanine at position 35 with glutamate, and a substitution of glutamine at position 41 with histidine. The gene encoding the amino acid sequence of SEQ ID NO: 2 is Hixy143A gene isolated from Humicola insolens. The xylosidase has a full length amino acid sequence of SEQ ID NO: 10.

According to another aspect of the present invention, there is provided a xylosidase comprising a modified amino acid sequence of SEQ ID NO: 2, wherein the modification is a substitution of phenylalanine at position 35 with glutamate. The gene encoding the amino acid sequence of SEQ ID NO: 2 is Hixy143A gene isolated from Humicola insolens. The xylosidase has a full length amino acid sequence of SEQ ID NO: 6.

According to an additional aspect of the present invention, there is provided a xylosidase comprising a modified amino acid sequence of SEQ ID NO: 2, wherein the modification is a substitution of glutamine at position 41 with histidine. The gene encoding the amino acid sequence of SEQ ID NO: 2 is Hixy143A gene isolated from Humicola insolens. The xylosidase has a full length amino acid sequence of SEQ ID NO: 8.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 shows the nucleotide sequence and the amino acid sequence of the wild type xylosidase Hixy143A;

FIG. 2 shows the mutagenic primer sequences for site-directed mutagenesis;

FIG. 3 shows the nucleotide sequence and the amino acid sequence of the F35E mutant;

FIG. 4 shows the nucleotide sequence and the amino acid sequence of the Q41H mutant;

FIG. 5 shows the nucleotide sequence and the amino acid sequence of the F35E/Q41H mutant; and

FIG. 6 shows the enzymatic activity analysis of the wild type Hixy143A and the three mutants.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

The xylosidase employed in the present invention is encoded by Hixy143A gene isolated from the themophilic fungus Humicola insolens Y1. According to previous studies, the optimal working condition of this xylosidase is at 50° C., pH 6.8. In the present invention, the Hixy143A gene was cloned into a vector and transformed into Pichia pastoris for protein expression. For improving the industrial application value of this xylosidase, the present invention analyzed its protein structure and chose some potential amino acids for modifications by site-directed mutagenesis so as to improve the enzymatic activity of the xylosidase. Based on the structural analysis, Phe35 and Gln41, which are both located in the active site of the xylosidase, were chosen for further modifications. By site-directed mutagenesis, Phe35 was singly mutated to glutamate as F35E mutant, while Gln41 was singly mutated to histidine as Q41H mutant. These two mutation sites were even combined into F35E/Q41H double mutant. The above mutations all successfully improved the enzymatic activity of the xylosidase.

The enzyme modification processes and the resulted xylosidase are described in detail as follows.

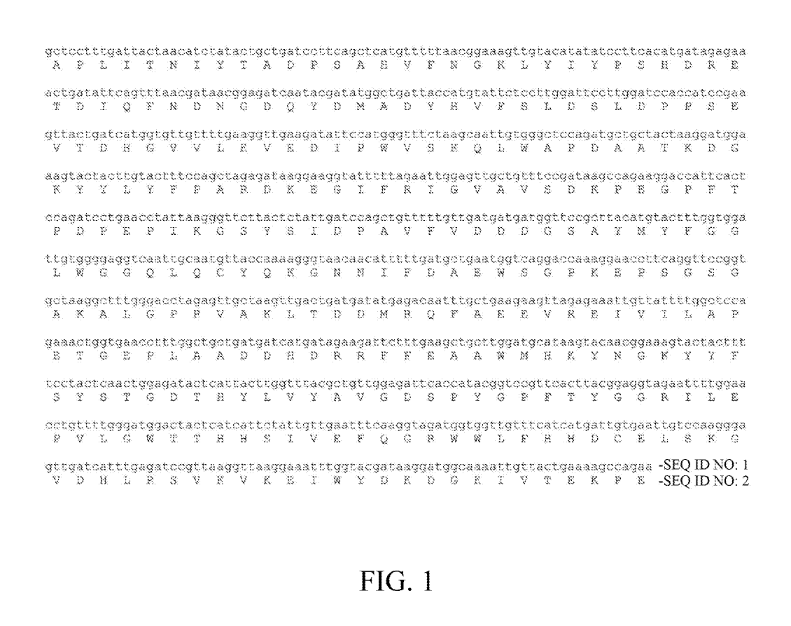

FIG. 1 shows the nucleotide sequence and the amino acid sequence of the wild type xylosidase Hixy143A, wherein the Hixy143A gene includes 978 base pairs (the nucleotide sequence was numbered as SEQ ID NO: 1) and encodes 326 amino acids (the amino acid sequence was numbered as SEQ ID NO: 2). First, the Hixy143A gene was cloned into pPICZαA vector by EcoRI and NotI. The plasmid DNA was linearized by PmeI and then transformed into Pichia pastoris. The transformants were selected by YPD plate with 0.1 mg/ml zeocin at 30° C. for 2 days. The selected clones were individually inoculated in YPD medium at 30° C. overnight and then amplified in BMGY medium at 30° C. overnight. Finally, the amplified cells were transferred to BMMY medium containing 0.5% methanol to induce the protein expression. The supernatants with induced proteins were collected by centrifugation for following analysis.

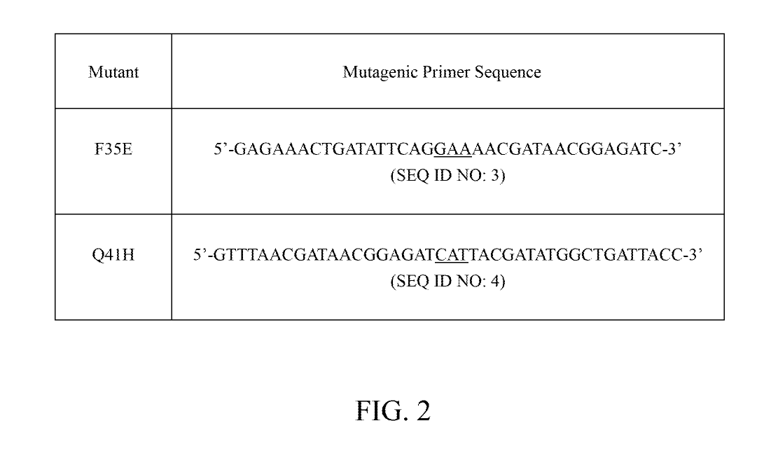

The three mutated genes of Hixy143A were obtained by site-directed mutagenesis. Particularly, these mutated sequences were obtained by polymerase chain reaction method using the wild type Hixy143A gene as the template and using the mutagenic primers shown in FIG. 2. F35E means the phenylalanine at position 35 was substituted with glutamate, and the mutagenic primer F35E was numbered as SEQ ID NO: 3. Q41H means the glutamine at position 41 was substituted with histidine, and the mutagenic primer Q41H was numbered as SEQ ID NO: 4. Therefore, the three mutated genes of Hixy143A obtained by site-directed mutagenesis in the present invention were F35E, Q41H and F35E/Q41H.

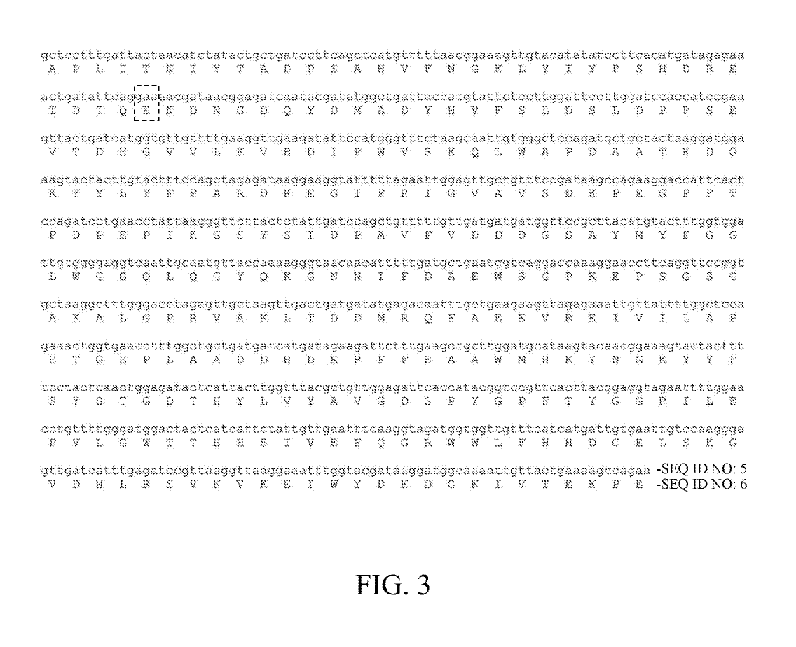

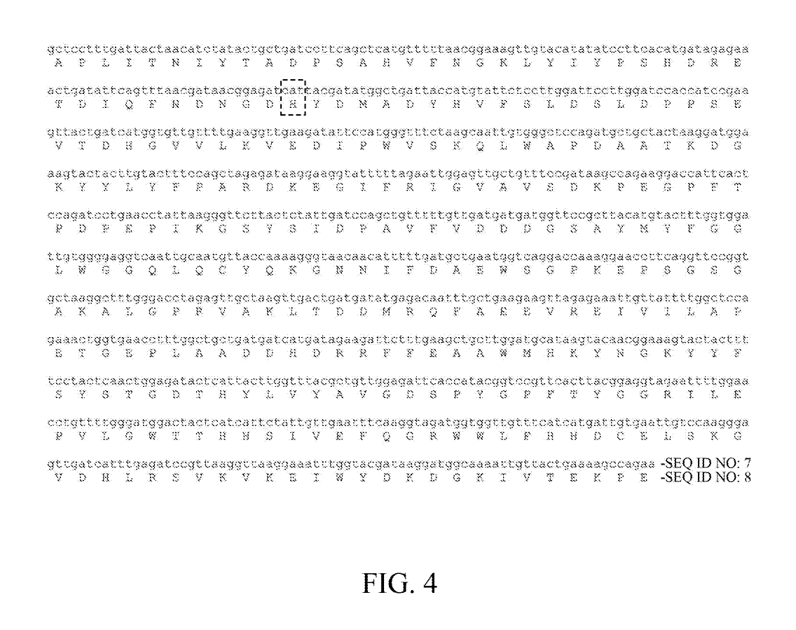

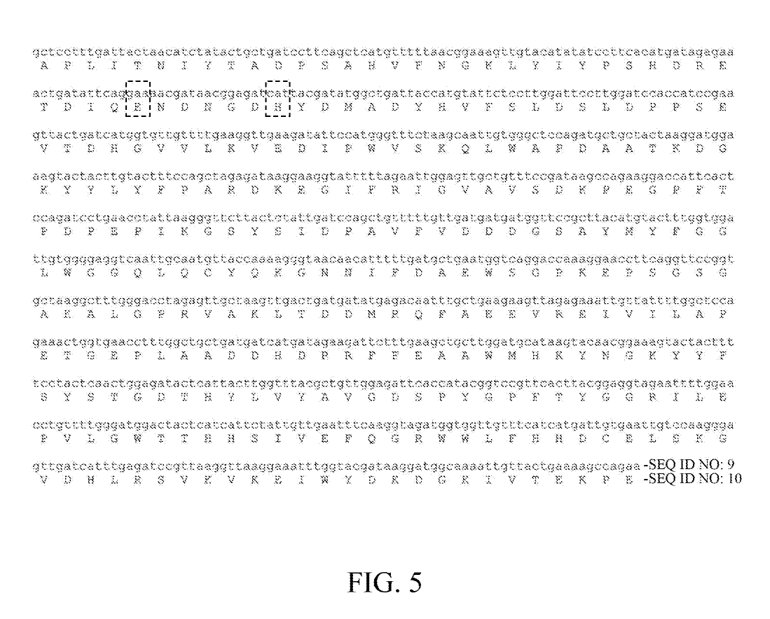

FIGS. 3 to 5 show the nucleotide sequences and the amino acid sequences of the three mutants. FIG. 3 shows the nucleotide sequence and the amino acid sequence of the F35E mutant, wherein the nucleotide sequence was numbered as SEQ ID NO: 5, the amino acid sequence was numbered as SEQ ID NO: 6, and the phenylalanine at position 35 was substituted with glutamate. FIG. 4 shows the nucleotide sequence and the amino acid sequence of the Q41H mutant, wherein the nucleotide sequence was numbered as SEQ ID NO: 7, the amino acid sequence was numbered as SEQ ID NO: 8, and the glutamine at position 41 was substituted with histidine. FIG. 5 shows the nucleotide sequence and the amino acid sequence of the F35E/Q41H mutant, wherein the nucleotide sequence was numbered as SEQ ID NO: 9, the amino acid sequence was numbered as SEQ ID NO: 10, and the phenylalanine at position 35 was substituted with glutamate and the glutamine at position 41 was substituted with histidine.

The original DNA template was removed by DpnI at 37° C. The three mutated genes were individually transformed into E. coli. The success of gene mutation was confirmed by DNA sequencing. Finally, the three successful mutated genes were separately transformed into P. pastoris and then induced for expressing the mutated proteins by the same method mentioned above. Afterwards, the wild type protein and the mutated proteins were further analyzed for their enzymatic activity.

The xylosidase activity analysis was determined by the measurement of released nitrophenol that is a chromogenic product from the hydrolysis of the substrate p-nitrophenyl-β-D-xylopyranoside by xylosidase and further calculated to determine the enzymatic activity of xylosidase. Basically, the reaction mixture composed of diluted protein sample and 5 mM p-nitrophenyl-β-D-xylopyranoside was incubated at 50° C. for 10 min. The reaction was then stopped by using 2 M Na₂CO₃. Finally, the absorption of OD410 nm was detected to determine the activity of xylosidase.

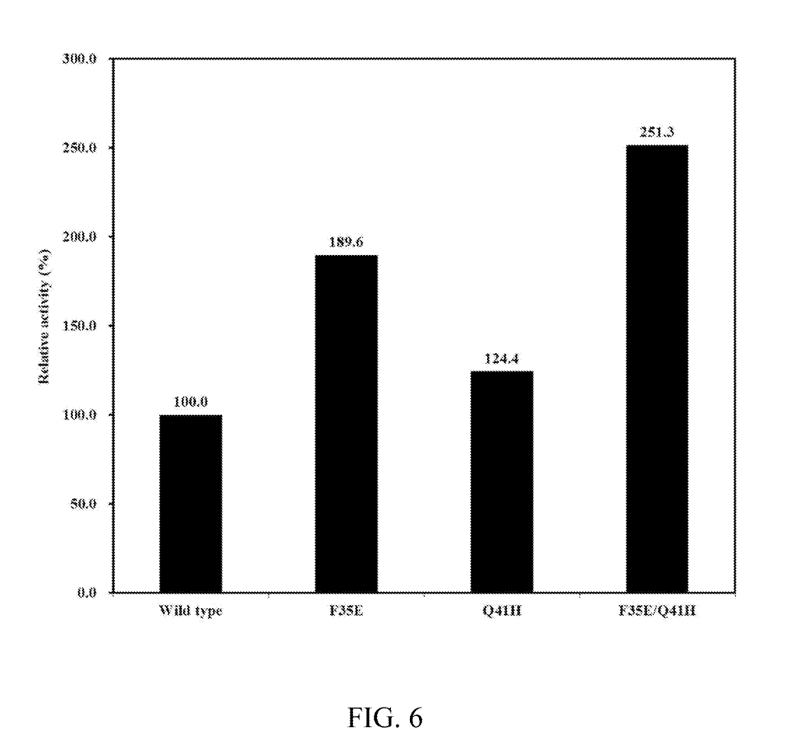

FIG. 6 shows the enzymatic activity analysis of the wild type Hixy143A and the three mutants. As shown in FIG. 6, under the same protein concentrations of these proteins, the single mutants F35E and Q41H both showed higher activities than did the wild type protein. When compared to the wild type protein, F35E mutant significantly increased the activity of nearly 90% while Q41H mutant increased the activity of about 20%. The double mutant F35E/Q41H showed notably increased activity to about 250%, which is much higher than the wild type protein and the single mutants. Besides, the protein expression levels of the mutants were similar to that of the wild type protein. Therefore, the total activities of these mutants F35E, Q41H and F35E/Q41H were also higher than that of the wild type protein, and the double mutant F35E/Q41H had the highest activity. It is clear that the three mutants provided in the present invention all have higher activities when compared to the wild type protein Hixy143A, that means the production cost of the xylosidase can be reduced, and thus the mutants have higher economic value of industrial application.

In conclusion, to improve the enzymatic activity of the xylosidase Hixy143A, the present invention chose some potential amino acids according to its structural analysis and further modified this enzyme by rational design. As a result, the three mutants including F35E, Q41H and F35E/Q41H all showed higher enzymatic activities compared to the wild type protein, and even had 2.5-fold increase. Therefore, the present invention successfully improves the enzymatic activity of the xylosidase and further increases its economic value of industrial application.

Nowadays, the application fields of the xylosidase and its related xylanase in industry are widespread. For feed industry, xylanolytic enzymes can be mixed with feed to help the digestion and absorption of the monogastric animals like pig and chicken by degrading feed materials with hemicellulose. As for paper and pulp industry, the enzymes also can reduce or replace the traditional toxic chemical method to reach the same result of bleaching. Besides, the enzymes provide assistances in the juice clearance and the saccharification step of brewing industry. As for biofuel production, xylosidase can degrade substrates to produce single sugars that can be utilized in fermentation by microorganisms. Thus, xylosidase can be widely used in various industries and has high economic value. The present invention modifies the xylosidase by genetic engineering, and the modified enzymes have significantly improved enzymatic activity, so the production cost of the xylosidase can be reduced to further improve the economic value of industrial application.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A xylosidase comprising a modified amino acid sequence of SEQ ID NO: 2, wherein the modification is a substitution of phenylalanine at position 35 with glutamate, and a substitution of glutamine at position 41 with histidine.

2. The xylosidase according to claim 1 wherein a gene encoding the amino acid sequence of SEQ ID NO: 2 is Hixy143A gene isolated from Humicola insolens.

3. The xylosidase according to claim 1 having a full length amino acid sequence of SEQ ID NO: 10.

4. A xylosidase comprising a modified amino acid sequence of SEQ ID NO: 2, wherein the modification is a substitution of phenylalanine at position 35 with glutamate.

5. The xylosidase according to claim 4 wherein a gene encoding the amino acid sequence of SEQ ID NO: 2 is Hixy143A gene isolated from Humicola insolens.

6. The xylosidase according to claim 4 having a full length amino acid sequence of SEQ ID NO: 6.

7. A xylosidase comprising a modified amino acid sequence of SEQ ID NO: 2, wherein the modification is a substitution of glutamine at position 41 with histidine.

8. The xylosidase according to claim 7 wherein a gene encoding the amino acid sequence of SEQ ID NO: 2 is Hixy143A gene isolated from Humicola insolens.

9. The xylosidase according to claim 7 having a full length amino acid sequence of SEQ ID NO: 8.


SEQUENCE LISTING (10 sequences)

SEQ ID NO: 1 - DNA, 978 bp, Humicola insolens
gctcctttgattactaacatctatactgctgatccttcagctcatgtttttaacggaaagttgtacatatatccttcacatgatagagaaactgatattcagtttaacgataacggagatcaatacgatatggctgattaccatgtattctccttggattccttggatccaccatccgaagttactgatcatggtgttgttttgaaggttgaagatattccatgggtttctaagcaattgtgggctccagatgctgctactaaggatggaaagtactacttgtactttccagctagagataaggaaggtatttttagaattggagttgctgtttccgataagccagaaggaccattcactccagatcctgaacctattaagggttcttactctattgatccagctgtttttgttgatgatgatggttccgcttacatgtactttggtggattgtggggaggtcaattgcaatgttaccaaaagggtaacaacatttttgatgctgaatggtcaggaccaaaggaaccttcaggttccggtgctaaggctttgggacctagagttgctaagttgactgatgatatgagacaatttgctgaagaagttagagaaattgttattttggctccagaaactggtgaacctttggctgctgatgatcatgatagaagattctttgaagctgcttggatgcataagtacaacggaaagtactacttttcctactcaactggagatactcattacttggtttacgctgttggagattcaccatacggtccgttcacttacggaggtagaattttggaacctgttttgggatggactactcatcattctattgttgaatttcaaggtagatggtggttgtttcatcatgattgtgaattgtccaagggagttgatcatttgagatccgttaaggttaaggaaatttggtacgataaggatggcaaaattgttactgaaaagccagaa

SEQ ID NO: 2 - protein, 326 aa, Humicola insolens
APLITNIYTADPSAHVFNGKLYIYPSHDRETDIQFNDNGDQYDMADYHVFSLDSLDPPSEVTDHGVVLKVEDIPWVSKQLWAPDAATKDGKYYLYFPARDKEGIFRIGVAVSDKPEGPFTPDPEPIKGSYSIDPAVFVDDDGSAYMYFGGLWGGQLQCYQKGNNIFDAEWSGPKEPSGSGAKALGPRVAKLTDDMRQFAEEVREIVILAPETGEPLAADDHDRRFFEAAWMHKYNGKYYFSYSTGDTHYLVYAVGDSPYGPFTYGGRILEPVLGWTTHHSIVEFQGRWWLFHHDCELSKGVDHLRSVKVKEIWYDKDGKIVTEKPE

SEQ ID NO: 3 - DNA, 36 bp, Artificial Sequence
  Synthetically generated primer
gagaaactgatattcaggaaaacgataacggagatc

SEQ ID NO: 4 - DNA, 41 bp, Artificial Sequence
  Synthetically generated primer
gtttaacgataacggagatcattacgatatggctgattacc

SEQ ID NO: 5 - DNA, 978 bp, Artificial Sequence
  Synthetically generated DNA encoding a modified      enzyme
gctcctttgattactaacatctatactgctgatccttcagctcatgtttttaacggaaagttgtacatatatccttcacatgatagagaaactgatattcaggaaaacgataacggagatcaatacgatatggctgattaccatgtattctccttggattccttggatccaccatccgaagttactgatcatggtgttgttttgaaggttgaagatattccatgggtttctaagcaattgtgggctccagatgctgctactaaggatggaaagtactacttgtactttccagctagagataaggaaggtatttttagaattggagttgctgtttccgataagccagaaggaccattcactccagatcctgaacctattaagggttcttactctattgatccagctgtttttgttgatgatgatggttccgcttacatgtactttggtggattgtggggaggtcaattgcaatgttaccaaaagggtaacaacatttttgatgctgaatggtcaggaccaaaggaaccttcaggttccggtgctaaggctttgggacctagagttgctaagttgactgatgatatgagacaatttgctgaagaagttagagaaattgttattttggctccagaaactggtgaacctttggctgctgatgatcatgatagaagattctttgaagctgcttggatgcataagtacaacggaaagtactacttttcctactcaactggagatactcattacttggtttacgctgttggagattcaccatacggtccgttcacttacggaggtagaattttggaacctgttttgggatggactactcatcattctattgttgaatttcaaggtagatggtggttgtttcatcatgattgtgaattgtccaagggagttgatcatttgagatccgttaaggttaaggaaatttggtacgataaggatggcaaaattgttactgaaaagccagaa

SEQ ID NO: 6 - protein, 326 aa, Artificial Sequence
  Sequence synthetically translated from       SEQ ID NO 5
APLITNIYTADPSAHVFNGKLYIYPSHDRETDIQENDNGDQYDMADYHVFSLDSLDPPSEVTDHGVVLKVEDIPWVSKQLWAPDAATKDGKYYLYFPARDKEGIFRIGVAVSDKPEGPFTPDPEPIKGSYSIDPAVFVDDDGSAYMYFGGLWGGQLQCYQKGNNIFDAEWSGPKEPSGSGAKALGPRVAKLTDDMRQFAEEVREIVILAPETGEPLAADDHDRRFFEAAWMHKYNGKYYFSYSTGDTHYLVYAVGDSPYGPFTYGGRILEPVLGWTTHHSIVEFQGRWWLFHHDCELSKGVDHLRSVKVKEIWYDKDGKIVTEKPE

SEQ ID NO: 7 - DNA, 978 bp, Artificial Sequence
  Synthetically generated DNA encoding a modified      enzyme
gctcctttgattactaacatctatactgctgatccttcagctcatgtttttaacggaaagttgtacatatatccttcacatgatagagaaactgatattcagtttaacgataacggagatcattacgatatggctgattaccatgtattctccttggattccttggatccaccatccgaagttactgatcatggtgttgttttgaaggttgaagatattccatgggtttctaagcaattgtgggctccagatgctgctactaaggatggaaagtactacttgtactttccagctagagataaggaaggtatttttagaattggagttgctgtttccgataagccagaaggaccattcactccagatcctgaacctattaagggttcttactctattgatccagctgtttttgttgatgatgatggttccgcttacatgtactttggtggattgtggggaggtcaattgcaatgttaccaaaagggtaacaacatttttgatgctgaatggtcaggaccaaaggaaccttcaggttccggtgctaaggctttgggacctagagttgctaagttgactgatgatatgagacaatttgctgaagaagttagagaaattgttattttggctccagaaactggtgaacctttggctgctgatgatcatgatagaagattctttgaagctgcttggatgcataagtacaacggaaagtactacttttcctactcaactggagatactcattacttggtttacgctgttggagattcaccatacggtccgttcacttacggaggtagaattttggaacctgttttgggatggactactcatcattctattgttgaatttcaaggtagatggtggttgtttcatcatgattgtgaattgtccaagggagttgatcatttgagatccgttaaggttaaggaaatttggtacgataaggatggcaaaattgttactgaaaagccagaa

SEQ ID NO: 8 - protein, 326 aa, Artificial Sequence
  Sequence synthetically translated from       SEQ ID NO 7
APLITNIYTADPSAHVFNGKLYIYPSHDRETDIQFNDNGDHYDMADYHVFSLDSLDPPSEVTDHGVVLKVEDIPWVSKQLWAPDAATKDGKYYLYFPARDKEGIFRIGVAVSDKPEGPFTPDPEPIKGSYSIDPAVFVDDDGSAYMYFGGLWGGQLQCYQKGNNIFDAEWSGPKEPSGSGAKALGPRVAKLTDDMRQFAEEVREIVILAPETGEPLAADDHDRRFFEAAWMHKYNGKYYFSYSTGDTHYLVYAVGDSPYGPFTYGGRILEPVLGWTTHHSIVEFQGRWWLFHHDCELSKGVDHLRSVKVKEIWYDKDGKIVTEKPE

SEQ ID NO: 9 - DNA, 978 bp, Artificial Sequence
  Synthetically generated DNA encoding a modified      enzyme
gctcctttgattactaacatctatactgctgatccttcagctcatgtttttaacggaaagttgtacatatatccttcacatgatagagaaactgatattcaggaaaacgataacggagatcattacgatatggctgattaccatgtattctccttggattccttggatccaccatccgaagttactgatcatggtgttgttttgaaggttgaagatattccatgggtttctaagcaattgtgggctccagatgctgctactaaggatggaaagtactacttgtactttccagctagagataaggaaggtatttttagaattggagttgctgtttccgataagccagaaggaccattcactccagatcctgaacctattaagggttcttactctattgatccagctgtttttgttgatgatgatggttccgcttacatgtactttggtggattgtggggaggtcaattgcaatgttaccaaaagggtaacaacatttttgatgctgaatggtcaggaccaaaggaaccttcaggttccggtgctaaggctttgggacctagagttgctaagttgactgatgatatgagacaatttgctgaagaagttagagaaattgttattttggctccagaaactggtgaacctttggctgctgatgatcatgatagaagattctttgaagctgcttggatgcataagtacaacggaaagtactacttttcctactcaactggagatactcattacttggtttacgctgttggagattcaccatacggtccgttcacttacggaggtagaattttggaacctgttttgggatggactactcatcattctattgttgaatttcaaggtagatggtggttgtttcatcatgattgtgaattgtccaagggagttgatcatttgagatccgttaaggttaaggaaatttggtacgataaggatggcaaaattgttactgaaaagccagaa

SEQ ID NO: 10 - protein, 326 aa, Artificial Sequence
  Sequence synthetically translated from       SEQ ID NO 9
APLITNIYTADPSAHVFNGKLYIYPSHDRETDIQENDNGDHYDMADYHVFSLDSLDPPSEVTDHGVVLKVEDIPWVSKQLWAPDAATKDGKYYLYFPARDKEGIFRIGVAVSDKPEGPFTPDPEPIKGSYSIDPAVFVDDDGSAYMYFGGLWGGQLQCYQKGNNIFDAEWSGPKEPSGSGAKALGPRVAKLTDDMRQFAEEVREIVILAPETGEPLAADDHDRRFFEAAWMHKYNGKYYFSYSTGDTHYLVYAVGDSPYGPFTYGGRILEPVLGWTTHHSIVEFQGRWWLFHHDCELSKGVDHLRSVKVKEIWYDKDGKIVTEKPE